(19) United States
(12) Patent Application Publication
(10) Pub. No.: US 2004/0185119 A1
(43) Pub. Date: Sep. 23, 2004

(54) Method and compositions for treating gastric hyperacidity while diminishing the likelihood of producing vitamin deficiency

(76) Inventor: Richard C. Theuer, Raleigh, NC (US)

Correspondence Address: HARNESS, DICKEY, & PIERCE, P.L.C, 7700 BONHOMME, STE 400, ST. LOUIS, MO, 63105, US

(21) Appl. No.: 10/766,227

(22) Filed: Jan. 28, 2004

Related U.S. Application Data
(60) Provisional application No. 60/450,246, filed on Feb. 26, 2003.

(51) Int. Cl. A61K 33/06; A61K 31/714; A61K 31/4439; A61K 31/375

(52) U.S. Cl. 424/682; 514/52; 514/338; 514/474

(57) ABSTRACT

A method for treatment of a human for gastric hyperacidity while diminishing the likelihood of producing vitamin deficiency is disclosed, the method comprising administering a therapeutically effective amount of one or more substances that neutralize or otherwise reduce gastric acid and administering an effective supplemental amount of one or more vitamins, wherein one of the one or more vitamins can be free Vitamin B₁₂. Oral dosage formulations comprising a therapeutically effective amount of one or more substances that neutralize or otherwise reduce gastric acid and an effective supplemental amount of one or more vitamins, and methods of making such oral dosage forms, also are disclosed.

### CROSS REFERENCE TO RELATED APPLICATION

This application claims the benefit of U.S. Provisional Application No. 60/450,246, filed Feb. 26, 2003.

### FIELD

This application relates generally to the treatment of humans with gastrointestinal disease characterized by gastric hyperacidity and, more particularly, to compositions and methods comprising a therapeutically effective amount of one or more substances that neutralize or otherwise inhibit gastric acid and an effective supplemental amount of one or more vitamins, and to methods for making these compositions.

### BACKGROUND

Sundry pharmaceutical interventions are employed to reduce gastric acidity in humans. However, such interventions can have negative nutritional effects. Reducing gastric acid can reduce the absorption of certain nutrients, leading to vitamin depletion. Accordingly, an unfilled need exists for methods and compositions for treating a human with gastric hyperacidity while diminishing the likelihood of producing vitamin deficiency.

### SUMMARY

Accordingly, the inventor herein has succeeded in devising compositions and methods for treating a human for gastric hyperacidity while diminishing the likelihood of producing vitamin deficiency. One method comprises administering a therapeutically effective amount of one or more substances that neutralize or otherwise reduce gastric acid and administering an effective supplemental amount of one or more vitamins. In various embodiments, the one of the one or more vitamins can include free Vitamin B₁₂.

Various embodiments of the present invention also can involve oral dosage formulations comprising a therapeutically effective amount of one or more substances that neutralize or otherwise reduce gastric acid and an effective supplemental amount of one or more vitamins.

In various embodiments, the present invention can also include methods of making such oral dosage forms. Such methods can comprise applying a coating comprising free Vitamin B₁₂ to an acceptable pharmaceutical preparation comprising one or more substances that neutralize or otherwise reduce gastric acid.

The present invention can also involve a method of making an oral dosage formulation comprising one or more proton pump inhibitors and one or more vitamins, said method comprising making a tablet comprising free Vitamin B₁₂, Vitamin C, and an acceptable pharmaceutical preparation comprising one or more proton pump inhibitors.

### DETAILED DESCRIPTION

The term “gastric hyperacidity” as used herein refers to medical conditions characterized by a relative or absolute excess of hydrochloric acid in the stomach, including without limitation hyperchlorhydria, gastroesophageal reflux disease, gastric or gastroduodenal ulcers, gastritis, hiatal hernia, and related gastrointestinal tract ailments accompanied by or related to a relative or absolute excess of hydrochloric acid in the stomach.

Gastric hyperacidity can be treated with pharmaceutical preparations comprising one or more pharmacologically active substances that neutralize or otherwise reduce gastric acid. Pharmacologically active substances that neutralize gastric acid are commonly referred to as “antacids”. The pharmacologically active substances most often prescribed to reduce gastric acid hypersecretion are classified generically as “proton pump inhibitors” and “histamine H₂-receptor antagonists”.

The term “proton pump inhibitor” as used herein refers to a pharmacologically active substance that binds irreversibly to H+/K+ ATPase, an enzyme found on the secretory surface of parietal cells of the stomach. This enzyme is essential for the final transport of hydrogen ions into the stomach by catalyzing the exchange of protons (H+) for potassium ions (K+). This enzyme is part of the “proton pump.” Inhibition of the proton pump by a proton pump inhibitor decreases the secretion of hydrochloric acid into the stomach and alters gastric pH.

Proton pump inhibitors include 2-pyridylmethylsulfinylbenzimidazole compounds differing in substituent groups. Examples without limitation are lansoprazole [The Merck Index, 12^{th} Ed., (p. 916) 1996], omeprazole [The Merck Index, 12^{th} Ed., (p. 1174) 1996], rapeprazole [The Merck Index, 12^{th} Ed., (p. 1392)] and leminoprazole, pantoprazole, and picoprazole, which are described by Depui et al. (U.S. Pat. No. 6,132,771). Therapeutically effective amounts of these proton pump inhibitors range from about 10 milligrams to about 40 milligrams.

Proton pump inhibitors used in the dosage forms known to those skilled in the art may be in neutral form or in the form of one or more alkaline salts, such as the magnesium, calcium, sodium, potassium or lithium salts. The proton pump inhibitors may be used in mixtures or in racemic form, the form of a substantially pure enantiomer thereof, the form of active isomers thereof, the form of derivatives, or the form of alkaline salts of the racemates, enantiomers, isomers and derivatives. An example of a racemic form is omeprazole. An example of an isomer is the omeprazole analog disclosed by Whittle et al. (U.S. Pat. No. 6,369,087). An example of an enantiomer is s-omeprazole, disclosed by Bergstrand et al. (U.S. Pat. No. 5,817,338) and others. An example of an alkaline salt is the magnesium salt of omeprazole disclosed by Bergstrand et al. (U.S. Pat. No. 5,817,338) and others.

The term “histamine H₂-receptor antagonist” as used herein refers to a pharmacologically active substance that reduces gastric acid output as a result of blockade of histamine H₂-receptors. The class of histamine H₂-receptor antagonists includes, without limitation, ranitidine [The Merck Index, 12^{th} Ed., (p. 1395) 1996], famotidine [The Merck Index, 12^{th} Ed., (p. 667) 1996], cimetidine [The Merck Index, 12^{th} Ed., (p. 383) 1996], nizatidine (Aymard, J. P., B. Aymard, et al. (1988). “Haematological adverse effects of histamine H2-receptor antagonists.” Med Toxicol Adverse Drug Exp 3(6): 430-48), and other compounds that inhibit gastric acid secretion by a similar mechanism. Therapeutically effective amounts of these histamine H₂-receptor antagonists are about 10 milligrams for famotidine, about 75 milligrams for ranitidine, about 200 mg for cimetidine, and about 300 mg for nizatidine.

The term “antacid” as used herein refers to a substance that neutralizes acid. The class of antacids includes, without limitation, alkali bicarbonates, such as sodium bicarbonate; carbonate, oxide and hydroxide compounds of calcium, magnesium and aluminum; and combinations thereof.

Vitamins include, without limitation, whether water-soluble or fat-soluble, ascorbic acid, thiamine, niacin, retinol palmitate, phytonadione, riboflavin, pyridoxine hydrochloride, cyanocobalamin, sodium ascorbate, cholecalciferol, nicotinic acid amide, calcium pantothenate, folic acid, biotin, inositol and choline.

The term “effective supplemental amount” of a vitamin as used herein refers to an amount of a vitamin that is within the range of the amounts used in fortified foods and dietary supplements and the amounts routinely provided on a daily basis for therapeutic restoration of biochemical normality in an individual with deficiency or low body stores of a vitamin. Fortified foods typically supply not less than 25% of the Recommended Dietary Allowance (“RDA”) where one is established. Therapeutic supplements can supply many times the RDA or the amount found in normal diets of those vitamins where an RDA is not established.

The term “Vitamin B₁₂” as used herein refers to all potentially biologically active cobalamins. Cobalamin is the general term used to describe a group of cobalt-containing compounds (corrinoids) that have a particular structure that contains the sugar ribose, phosphate, and a base (5,6-benzimidazole) attached to the corrin ring. Vitamin B₁₂ functions as a coenzyme for critical metabolic reactions in the mammalian body.

The term “free Vitamin B₁₂” as used herein refers to Vitamin B₁₂ not bound to protein. Fortified foods and oral dosage forms comprising Vitamin B₁₂ customarily contain free Vitamin B₁₂. Cyanocobalamin is a purified substance and a widely used free Vitamin B₁₂. Cyanocobalamin is the free Vitamin B₁₂ most commonly incorporated into vitamin and nutritional supplements and fortified breakfast cereals. Analogs of cyanocobalamin which differ only in the β-ligand of the cobalt also are free Vitamin B₁₂. Examples of said analogs include without limitation hydroxocobalamin, methylcobalamin, aquocobalamin, acetatocobalamin, nitrocobalamin, and sufitocobalamin, substances well known to those skilled in the art.

The term “effective supplemental amount of free Vitamin B₁₂” as used herein includes the range from 0.5 microgram, or 25% of the adult Estimated Average Requirement (EAR), to about 5 milligrams. The Estimated Average Requirement (EAR) of Vitamin B₁₂ for adult men and women 19 to 50 years of age is 2 micrograms (IOM, 2000a). The RDA (Recommended Dietary Allowance) of Vitamin B₁₂ for adult men and women is 2.4 micrograms. Therapeutic Vitamin B₁₂ supplements for oral administration contain 2.5 milligrams or more of Vitamin B₁₂.

The term “Vitamin C” as used herein refers to ascorbic acid [The Merck Index, 12^{th} Ed., (p. 139) 1996] and dehydroascorbic acid [The Merck Index, 12^{th} Ed., (p. 485) 1996], and also alkali and alkaline salts and derivatives of these acids. Examples of said alkali and alkaline salts include, without limitation, sodium ascorbate [The Merck Index, 12^{th} Ed., (p. 1471) 1996] and calcium ascorbate [The Merck Index, 12^{th} Ed., (p. 270) 1996]. An example of said derivatives is ascorbyl palmitate.

The term “effective supplemental amount of Vitamin C” as used herein includes the range from range from about 20 milligrams to about 2 grams. The Estimated Average Requirement (EAR) of Vitamin C for adult men 19 to 50 years of age is 75 milligrams. The RDA (Recommended Dietary Allowance) of Vitamin C for adult men is 90 milligrams. (Institute of Medicine, 2000b. Dietary reference intakes for Vitamin C, Vitamin E, Selenium, and Carotenoids. A report of the Panel on Dietary Antioxidants and Related Compounds, Subcommittees on Upper Reference Levels of Nutrients and Interpretation and Uses of Dietary Reference Intakes, and the Standing Committee on the Scientific Evaluation of Dietary Reference Intakes. Food and Nutrition Board, Institute of Medicine. Pp. 146-147). Therapeutic dose oral Vitamin C supplements contain 1000 milligrams or more of Vitamin C.

The term “unit dose” as used herein refers to the prescribed amount of each dosage in a package. The term “blister pack” as used herein refers to a unit-dose package commonly constructed from a formed cavity containing one or more individual doses. The term “strip pack” as used herein refers to a package used to protect solid dose pharmaceutical products, and to provide relatively inexpensive protection for individual dosages.

The term “coating” as used herein refers to material comprising one or more substances, said material resulting from a process of applying the one or more substances to a substrate. This technology is well known to those skilled in the art. See, for example, Porter, “Coating of Pharmaceutical Dosage Forms,” Chapter 46, pages 894-902, in “Remington: The Science and Practice of Pharmacy.” Twentieth Edition, A. R. Gennaro et al., Editors. Lippincott-Williams&Wilkins, 2000, which is hereby incorporated in whole by reference. Porter teaches that basically there are four major techniques for applying coatings to pharmaceutical dosage forms: sugar coating, film coating, microencapsulation and compression coating.

The term “an acceptable pharmaceutical preparation comprising one or more proton pump inhibitors” as used herein refers to a pharmaceutical preparation that protects an acid-labile proton pump inhibitor from premature dissolution in the stomach. 2-Pyridylmethylsulfinylbenzimidazole compounds are susceptible to degradation or transformation in acid reacting or neutral media. Omeprazole is also sensitive to moisture.

It is well known to those skilled in the art that proton pump inhibitor formulations must be carefully constructed to prevent or minimize contact of the active drug with the acidic gastric contents and yet to be able to dissolve rapidly in the small intestine.

Examples of acceptable pharmaceutical preparations comprising one or more proton pump inhibitors include, without limitation, tablets and pellets.

Therapeutic agents that inhibit acid secretion and thereby increase gastric pH can interfere with the release and absorption of protein-bound Vitamin B₁₂ (Carmel, 1995) (Schenk et al. Aliment. Pharmacol. Ther. 10:541-545, 1996). Ruscin et al. (Ann. Pharmacother. 36:812-816, 2002) described a case of Vitamin B₁₂ deficiency associated with long-term use of a histamine H₂-receptor antagonist and a proton pump inhibitor.

Reviewers of the effects of gastric acid-suppressing drugs on Vitamin B₁₂ have recommended that healthcare workers should be made aware of the potential for Vitamin B₁₂ malabsorption, should consider periodic testing of Vitamin B₁₂ status, and should treat patients with signs of Vitamin B₁₂ deficiency or with low blood levels of Vitamin B₁₂. However, no suggestion has been made to incorporate a source of free Vitamin B₁₂ into the standard regime of proton pump inhibitor therapy or histamine H₂-receptor antagonist therapy.

Proton pump inhibitors reduce gastric ascorbic acid levels and increase gastric nitrite levels in H. pylori-positive individuals (Mowat et al., Best Pract. Res. Clin. Gastroenterol. 15:523-537, 2001).

The present invention, in various embodiments, can involve providing a therapeutically effective amount of one or more substances that neutralize or otherwise reduce gastric acid and, in addition, providing an effective supplemental amount of one or more vitamins. The one or more vitamins can be in the oral dosage formulation with the one or more substances that neutralize or otherwise reduce gastric acid or in a separate oral dosage formulation. The one or more vitamins can include Vitamin B₁₂. In embodiments in which Vitamin B₁₂ is included, the Vitamin B₁₂ can be in a formulation that provides an effective amount of free Vitamin B₁₂. Effective amounts of free Vitamin B₁₂ can include from about 0.5 micrograms to about 5 milligrams; from about 2 micrograms to about 2 milligrams, from about 5 micrograms to about 5 milligrams, from about 50 micrograms to about 500 milligrams or from about 100 micrograms to about 250 micrograms.

The one or more vitamins can also include Vitamin C. Effective amounts of Vitamin C can also be included with the one or more substances that neutralize or otherwise reduce gastric acid or in a separate dosage formulation. Such effective supplemental amounts of Vitamin C can include from about 20 milligrams to about 2 grams; from about 100 milligrams to about 1 gram or from about 200 milligrams to about 1 gram.

Other vitamins that can be included with the one or more substances that neutralize or otherwise reduce gastric acid include folic acid.

In various embodiments, the invention can involve units of a first oral dosage formulation comprising one or more substances that neutralize or otherwise reduce gastric acid and units of a second dosage formulation comprising one or more vitamins packaged together in unit-dose packaging.

In various embodiments, the invention can involve providing one or more substances that neutralize or otherwise reduce gastric acid and one or more vitamins in a single oral dosage formulation. The formulation can be any acceptable oral pharmaceutical dosage formulation known to those skilled in the art, including without limitation tablets and capsules.

In various embodiments, the invention can involve providing therapeutically effective amounts of one or more proton pump inhibitors and an amount of free Vitamin B₁₂.

In various embodiments, the invention can involve providing therapeutically effective amounts of one or more proton pump inhibitors, an amount of free Vitamin B₁₂, and an amount of Vitamin C.

In various embodiments, the invention can involve an oral dosage formulation comprising one or more proton pump inhibitors and one or more vitamins.

In various embodiments, the invention can involve a method of making an oral dosage formulation comprising one or more proton pump inhibitors and one or more vitamins, said method comprising the application of a coating comprising free Vitamin B₁₂ to an acceptable pharmaceutical preparation comprising one or more proton pump inhibitors. Acceptable pharmaceutical preparations include, without limitation, tablets, enterically coated pellets, film-coated enterically coated pellets, and capsules.

In various embodiments, the invention can involve a method of making an oral dosage formulation comprising one or more proton pump inhibitors and one or more vitamins, said method comprising making a tablet comprising free Vitamin B₁₂, Vitamin C, and an acceptable pharmaceutical preparation comprising one or more proton pump inhibitors.

The following examples are further illustrative of the present invention, but it is understood that the invention is not limited thereto.

### EXAMPLE 1

This example illustrates unit-dose packaging. One or more proton pump inhibitors can be provided in a first oral dosage formulation and free Vitamin B₁₂ can be provided in a second oral dosage formulation. Said first oral dosage form and said second oral dosage form can be co-packaged in a blister pack. Said first oral dosage form can be omeprazole 20 mg delayed-release capsules distributed by Kremers Urban, Mequon, Wis. 53092, identified as NDC (National Drug Code) 62175-118-32. Said second oral dosage form can be Kroger Vitamin B₁₂ 500 mcg Dietary Supplement, pink scored tablets distributed by the Kroger Co., Cincinnati, Ohio 45202, identified by the UPC (Universal Product Code) 0-11110-79860-2. The ingredients of said second oral dosage form are lactose, dicalcium phosphate, corn starch, magnesium stearate, cyanocobalamin, and Red 40 lake. In addition to unit-dose convenience, the blister pack provides moisture protection to the omeprazole capsules until the point of actual administration.

### EXAMPLE 2

This example illustrates a tablet made with 1% cyanocobalamin and lansoprazole pellets. A tablet comprising the proton pump inhibitor lansoprazole and cyanocobalamin can be made with enteric coating layered pellets, said pellets comprising lansoprazole and prepared according to Example 5 of Depui et al. (U.S. Pat. No. 6,132,771). Lansoprazole can be sprayed onto sugar sphere seeds in a fluid bed apparatus from a water suspension containing dissolved hydroxypropyl methylcellulose and sodium lauryl sulfate. The so-prepared core material can then be covered with a separating layer made with a hydroxypropyl cellulose solution containing talc and magnesium stearate. The so-prepared core material with a separating layer can then covered with an enteric coating consisting of methacrylic acid copolymer, mono- and diglycerides, triethyl citrate and polysorbate 80 sprayed on in a fluid bed apparatus. The enteric coating layered pellets thus prepared according to Example 5 of Depui et al. will contain approximately 21% lansoprazole.

Said enteric coating layered pellets can be dry mixed with microcrystalline cellulose, crosslinked polyvinylpyrrolidone, sodium stearyl fumarate, and a 1% cyanocobalamin material in the following proportions.

i. enteric coating layered pellets, 47 g;

ii. microcrystalline cellulose, 280 g

iii. crosslinked polyvinylpyrrolidone, 5 g

iv. sodium stearyl fumarate, 0.5 g

v. 1% cyanocobalamin material, 27.5 g

Said 1% cyanocobalamin material is a free-flowing pink powder of almost spherical particles or agglomerates and consists of cyanocobalamin USP in a matrix of maltodextrin buffered with citric acid and trisodium citrate (available from BASF Corp., Mount Olive, N.J. 07828).

The blended material can be compressed into tablets weighing approximately 360 mg that contain approximately 10 mg of lansoprazole and approximately 250 mcg of cyanocobalamin (with a 10% overage).

The tablets so produced can be film-coated in a conventional manner if so desired.

### EXAMPLE 3

This example illustrates a tablet made with 1% cyanocobalamin, ascorbic acid for direct compression, and lansoprazole. A tablet comprising the proton pump inhibitor lansoprazole and the vitamins ascorbic acid and cyanocobalamin can be made with the enteric coating layered pellets of Example 2 above prepared according to Example 5 of Depui et al. (U.S. Pat. No. 6,132,771). The enteric coating layered pellets prepared according to Example 5 of Depui et al. will contain approximately 21% lansoprazole.

Said enteric coating layered pellets can be dry mixed with microcrystalline cellulose, crosslinked polyvinylpyrrolidone, 1% cyanocobalamin material, 97% ascorbic acid for direct compression, and magnesium stearate, in the following proportions.

i. enteric coating layered pellets, 47 g;

ii. 1% cyanocobalamin material, 27.5 g

iii. 97% ascorbic acid for direct compression, 283.5 g

iv. microcrystalline cellulose, 237 g

v. crosslinked polyvinylpyrrolidone, 12 g

vi. magnesium stearate, 3 g

Said 97% ascorbic acid for direct compression is a white or off-white fine granular powder and consists of 97% ascorbic acid USP and 3% food starch (BASF Corp., Mount Olive, NJ 07828).

The blended material can be compressed into tablets weighing approximately 610 mg that contain approximately 10 mg of lansoprazole, approximately 250 mcg of cyanocobalamin (with a 10% overage) and approximately 250 mg of ascorbic acid (with a 10% overage).

The tablets so produced can be film-coated in a conventional manner if so desired.

### EXAMPLE 4

This example illustrates a tablet made with 1% cyanocobalamin, sodium ascorbate for direct compression, and lansoprazole pellets. A tablet comprising the proton pump inhibitor lansoprazole and the vitamins ascorbic acid and cyanocobalamin can be made with the enteric coating layered pellets of Example 2 above prepared according to Example 5 of Depui et al. (U.S. Pat. No. 6,132,771). The enteric coating layered pellets prepared according to Example 5 of Depui et al. will contain approximately 21% lansoprazole.

Said enteric coating layered pellets can be dry mixed with microcrystalline cellulose, crosslinked polyvinylpyrrolidone, 1% cyanocobalamin material, 99% sodium ascorbate for direct compression, 97% ascorbic acid for direct compression, and magnesium stearate, in the following proportions.

i. enteric coating layered pellets, 47 g;

ii. 1% cyanocobalamin material, 27.5 g

iii. 99% sodium ascorbate for direct compression, 157.5 g

iv. 97% ascorbic acid for direct compression, 142 g

v. microcrystalline cellulose, 221 g

vi. crosslinked polyvinylpyrrolidone, 12 g

vii. magnesium stearate, 3 g

Said 99% sodium ascorbate for direct compression is a white to yellowish fine granular powder and consists of 99% sodium ascorbate USP and 1% food starch (BASF Corp., Mount Olive, N.J. 07828).

The blended material can be compressed into tablets weighing approximately 610 mg that contain approximately 10 mg of lansoprazole, approximately 250 mcg of cyanocobalamin (with a 10% overage) and approximately 250 mg of ascorbic acid (with a 10% overage).

The tablets so produced can be film-coated in a conventional manner if so desired.

### EXAMPLE 5

This example illustrates an omeprazole tablet made with cyanocobalamin in the film coating layer. Tablets comprising the proton pump inhibitor salt magnesium omeprazole can be made according to the method of Example 1 of Bergstrand et al. (U.S. Pat. No. 6,428,810). The tablets can be film-coated in a conventional manner with the following tablet-coating solution containing cyanocobalamin USP.

Per 10,000 tablets:

i. Hydroxypropyl methylcellulose, 100 g

ii. Polyethylene glycol 6000, 25 g

iii. Titanium dioxide, 25 g

iv. Cyanocobalamin, 1.1 g

v. Purified water, 850 g

The film-coated tablets provide approximately 20 mg of omeprazole and approximately 100 mcg of cyanocobalamin (with a 10% overage).

### EXAMPLE 6

This example illustrates another omeprazole tablet made with cyanocobalamin in the film coating layer. A granulation comprising the proton pump inhibitor omeprazole can be made according to the method of Example 4 of Chen et al. (U.S. Pat. No. 6,174,548) and then used to make tablets. The resulting tablets can be given an enteric coating according to the method of Example 1 of Chen et al. The seal coat disclosed in Example 1 of Chen et al. can be modified to incorporate free Vitamin B₁₂. Said seal coat can be applied to said enteric coated tablets as follows.

i. Enteric coated tablets, 146.0 g

ii. Opadry II pink, 4.5 g

iii. Cyanocobalamin USP, 0.22 g

iv. Water 450.0 g

The seal coat solution can be applied to the enteric coated omeprazole tablets using a perforated pan coater as disclosed by Chen et al. The finished seal-coated tablets provide 20 mg of omeprazole and 200 micrograms of cyanocobalamin with a 10% overage.

### EXAMPLE 7

This example illustrates a capsule made with enteric coating layered pellets with over-coating comprising cyanocobalamin. A capsule comprising magnesium omeprazole and cyanocobalamin can be made with enteric coating layered pellets comprising magnesium omeprazole and prepared according to Example 1 of Depui et al. (U.S. Pat. No. 6,132,771). Magnesium omeprazole can be sprayed onto sugar sphere seeds in a fluid bed apparatus from a water suspension containing dissolved hydroxypropyl methylcellulose. The so-prepared core material can then be covered with a separating layer made with a hydroxypropyl cellulose solution containing talc and magnesium stearate. The so-prepared core material with a separating layer can then covered with an enteric coating consisting of methacrylic acid copolymer, mono- and diglycerides, triethyl citrate and polysorbate 80 sprayed on in a fluid bed apparatus. The enteric coating layered pellets prepared according to Example 1 of Depui et al. will contain approximately 14% omeprazole.

The enteric coating layered pellets so prepared can be provided with an over-coating layer comprising cyanocobalamin in the following manner:

i. Enteric coating layered pellets, 20 kg

ii. Hydroxypropyl methylcellulose, 238 g

iii. Cyanocobalamin USP, 16 g

iv. Magnesium stearate, 7 g

v. Purified water, 6.56 kg

The enteric coating layered pellets can be coated with the hydroxypropyl methylcellulose and cyanocobalamin solution containing magnesium stearate in a fluid bed apparatus.

The so-prepared enteric coating layered pellets with an over-coating layer comprising cyanocobalamin can be filled into hard gelatin capsules with a target fill weight of 142 mg which will provide approximately 20 mg of omeprazole and 100 mcg of free Vitamin B₁₂ with a 10% overage.

All references cited in this specification are hereby incorporated by reference. Any discussion of references cited herein is intended merely to summarize the assertions made by their authors and no admission is made that any reference or portion thereof constitutes relevant prior art. Applicant reserves the right to challenge the accuracy and relevance of the cited references.

While certain preferred and alternative embodiments of the invention have been set forth for purposes of disclosing the invention, modifications to the disclosed embodiments may occur to those skilled in the art. The description of the invention is merely exemplary in nature and, thus, variations that do not depart from the gist of the invention are intended to be within the scope of the invention. Such variations are not to be regarded as a departure from the spirit and scope of the invention. Accordingly the appended claims are intended to cover all embodiments of the invention and modifications thereof which do not depart from the spirit and scope of the invention.

What I claim is:

1. A method for treating a human for gastric hyperacidity while diminishing the likelihood of producing vitamin deficiency by the treatment, said method comprising administering a therapeutically effective amount of one or more substances that neutralize or otherwise reduce gastric acid and administering an effective supplemental amount of one or more vitamins

2. The method of claim 1, wherein the one or more substances that neutralize or otherwise reduce gastric acid are selected from the group consisting of antacids, histamine H₂-receptor antagonists, and proton pump inhibitors.

3. The method of claim 5, wherein said histamine H₂-receptor antagonists are selected from the group consisting of ranitidine, famotidine, cimetidine, and nizatidine.

4. The method of claim 2, wherein said proton pump inhibitors are 2-pyridylmethylsulfinylbenzimidazole compounds differing in substituent groups.

5. The method of claim 4, wherein said proton pump inhibitors are selected from the group consisting of the compounds lansoprazole, leminoprazole, omeprazole, pantoprazole, rabeprazole, and picoprazole, racemates thereof, enantiomers thereof, isomers thereof, derivatives thereof, and alkaline salts of said compounds, said racemates, said enantiomers, said isomers, and said derivatives.

6. The method of claim 1, wherein the one or more vitamins is selected from the group consisting of Vitamin B₁₂ Vitamin C and combinations thereof.

7. The method of claim 6, wherein the Vitamin B₁₂ comprises free Vitamin B₁₂.

8. The method of claim 7, wherein free Vitamin B₁₂ is selected from the group consisting of cyanocobalamin, hydroxocobalamin, methylcobalamin, aquocobalamin, acetatocobalamin, nitrocobalamin, and sufitocobalamin.

9. The method of claim 7, wherein free Vitamin B₁₂ is cyanocobalamin.

10. The method of claim 7, wherein the effective supplemental amount of free Vitamin B₁₂ is 0.1 micrograms to 5 milligrams.

11. The method of claim 6, wherein the one or more Vitamins further comprises Vitamin C.

12. The method of claim 10, wherein the Vitamin C is selected from the group consisting of ascorbic acid, an alkali or alkaline salt of ascorbic acid, or ascorbyl palmitate.

13. The method of claim 11, wherein the effective supplemental amount of Vitamin C is 7.5 milligrams to 2 grams.

14. The method of claim 1, wherein the one or more substances that neutralize or otherwise reduce gastric acid are provided in a first oral dosage form and one or more vitamins are provided in a second oral dosage form.

15. The method of claim 14, wherein the units of said first oral dosage form intended for daily ingestion and the units of said second dosage form intended for daily ingestion are packaged together in unit-dose packaging.

16. The method of claim 1, wherein the one or more substances that neutralize or otherwise reduce gastric acid and the one or more vitamins comprise a single oral dosage form.

17. An oral dosage formulation comprising a therapeutically effective amount of one or more substances that neutralize or otherwise reduce gastric acid and an effective supplemental amount of one or more vitamins.

18. The formulation of claim 17 wherein the one or more substances that neutralize or otherwise reduce gastric acid are selected from the group consisting of antacids, histamine H₂-receptor antagonists, and proton pump inhibitors.

19. The formulation of claim 18, wherein said histamine H₂-receptor antagonists are selected from the group consisting of ranitidine, famotidine, cimetidine, and nizatidine.

20. The formulation of claim 18 wherein the one or more proton pump inhibitors are selected from the group consisting of the compounds lansoprazole, leminoprazole, omeprazole, pantoprazole, pariprazole, rabeprazole and picoprazole, racemates thereof, enantiomers thereof, isomers thereof, derivatives thereof, and alkaline salts of said compounds, said racemates, said enantiomers, said isomers, and said derivatives.

21. The formulation of claim 17 wherein the one or more vitamins is selected from the group consisting of free Vitamin B₁₂, Vitamin C and combinations thereof.

22. The formulation of claim 21 wherein the one or more vitamins comprises free Vitamin B₁₂.

23. The formulation of claim 22 wherein free Vitamin B₁₂ is selected from the group consisting of cyanocobalamin, hydroxocobalamin, methylcobalamin, aquocobalamin, acetatocobalamin, nitrocobalamin, and sufitocobalamin.

24. The formulation of claim 23, wherein free Vitamin B₁₂ is cyanocobalamin.

25. The formulation of claim 22, wherein the effective supplemental amount of free Vitamin B₁₂ is 0.1 micrograms to 5 milligrams.

26. The formulation of claim 21 wherein the one or more vitamins comprises Vitamin C.

27. The formulation of claim 26 wherein Vitamin C is selected from the group consisting of ascorbic acid, an alkali or alkaline salt of ascorbic acid, or ascorbyl palmitate.

28. The formulation of claim 26 wherein the effective supplemental amount of free Vitamin C is 5 milligrams to 2 grams.

29. A method of making an oral dosage formulation comprising a therapeutically effective amount of one or more substances that neutralize or otherwise reduce gastric acid and an effective supplemental amount of one or more vitamins, said method comprising applying a coating comprising free Vitamin B₁₂ to an acceptable pharmaceutical preparation comprising one or more substances that neutralize or otherwise reduce gastric acid.

30. The method of claim 29 wherein the one or more substances that neutralize or otherwise reduce gastric acid are selected from the group consisting of antacids, histamine H₂-receptor antagonists, and proton pump inhibitors.

31. The method of claim 30, wherein said histamine H₂-receptor antagonists are selected from the group consisting of ranitidine, famotidine, cimetidine, and nizatidine.

32. The method of claim 30, wherein said proton pump inhibitors are selected from the group consisting of the compounds lansoprazole, leminoprazole, omeprazole, pantoprazole, pariprazole, rabeprazole and picoprazole, racemates thereof, enantiomers thereof, isomers thereof, derivatives thereof, and alkaline salts of said compounds, said racemates, said enantiomers, said isomers, and said derivatives.

33. The method of claim 29 wherein free Vitamin B₁₂ is selected from the group consisting of cyanocobalamin, hydroxocobalamin, methylcobalamin, aquocobalamin, acetatocobalamin, nitrocobalamin, and sufitocobalamin.

34. The method of claim 33, wherein free Vitamin B₁₂ is cyanocobalamin.

35. The method of claim 33, wherein the effective supplemental amount of free Vitamin B₁₂ is 0.1 micrograms to 5 milligrams.

36. The method of claim 29, wherein said pharmaceutical preparation to which said coating is applied is a tablet.

37. The method of claim 29, wherein said pharmaceutical preparation to which said coating is applied is an enterically coated pellet.

38. The method of claim 29, wherein said pharmaceutical preparation to which said coating is applied is an enterically coated pellet that has been film-coated.

39. The method of claim 29, wherein said pharmaceutical preparation to which said coating is applied is a capsule.

40. The method of claim 29, wherein the one or more vitamins comprises Vitamin C.

41. A method of making an oral dosage formulation comprising one or more proton pump inhibitors and one or more vitamins, said method comprising making a tablet comprising free Vitamin B₁₂, Vitamin C, and an acceptable pharmaceutical preparation comprising one or more proton pump inhibitors.

42. The method of claim 35, wherein said pharmaceutical preparation is an enterically coated pellet.

43. The method of claim 35, wherein said pharmaceutical preparation is an enterically coated pellet that has been film-coated.
